(19) United States
(12) Patent Application Publication
(10) Pub. No.: US 2007/0003604 A1
(43) Pub. Date: Jan. 4, 2007

(54) Tissue coverings bearing customized tissue images

(75) Inventor: Jeffrey W. Jones, Robertson, WY (US)

Correspondence Address: Kenton R. Mullins;Stout, Uxa, Buyan & Mullins, LLP, Suite 300, 4 Venture, Irvine, CA, 92618, US

(73) Assignee: BioLase Technology, Inc, Irvine, CA (US)

(21) Appl. No.: 11/454,627

(22) Filed: Jun. 16, 2006

Related U.S. Application Data
(60) Provisional application No. 60/691,503, filed on Jun. 16, 2005.

(51) Int. Cl. A61K 9/70

(52) U.S. Cl. 424/443

(57) ABSTRACT

Tissue coverings customized with images of underlying tissue are disclosed. Wounds, unwanted features, blemishes and the like may be camouflaged or masked by the tissue coverings, which can include photographic images of a patient's underlying tissue.

### CROSS-REFERENCE TO RELATED APPLICATIONS

This application claims the benefit of U.S. Provisional Application No. 60/691,503, filed Jun. 16, 2005 and entitled TISSUE COVERINGS BEARING CUSTOMIZED TISSUE IMAGES (Att. Docket B19892PR), the entire contents of all which are hereby incorporated by reference.

### BACKGROUND OF THE INVENTION

Various adhesive structures and image printing apparatuses have existed in the prior art, including wound covering implements having adhesive peripheral areas and centrally located, sterile, wound-contacting bandage portions. Surfaces of such structures or carrier materials facing away from the wound contacting bandage portions have been printed with colors or images. Examples of applications of images to carrier materials are disclosed in U.S. Pat. Nos. 6,297,420, 6,455,752, and 6,512,160.

### SUMMARY OF THE INVENTION

An embodiment of the present invention comprises a device for contacting or camouflaging a target area of tissue. The device may comprise a carrier constructed to be applied in proximity to and to at least partially cover the target area of tissue. The device further may comprise a surface coupled to the carrier and constructed with an image of the target area of tissue. Another embodiment of the present invention comprises a device for covering an unwanted feature on an application area of skin of a patient. The device may be formed of a carrier sheet having a first side on which is disposed a modified image of the application area. A second side of the carrier sheet may have disposed thereon an adhesive backing adapted to being adhered to the application area.

The present invention further comprises a method of covering a target area of tissue. An implementation of the method comprises capturing a source image and generating a modification of the source image to create a modified image. The modified image may be transferred to the target area of tissue.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112.

Any feature or combination of features described herein are included within the scope of the present invention provided that the features included in any such combination are not mutually inconsistent as will be apparent from the context, this specification, and the knowledge of one skilled in the art. In addition, any feature or combination of features may be specifically excluded from any embodiment of the present invention. For purposes of summarizing the present invention, certain aspects, advantages and novel features of the present invention are described. Of course, it is to be understood that not necessarily all such aspects, advantages or features will be embodied in any particular implementation of the present invention. Additional advantages and aspects of the present invention are apparent in the following detailed description and claims that follow.

### BRIEF DESCRIPTION OF THE DRAWINGS

FIG. 1 is a block diagram of an embodiment of a device for treating a target area of tissue;

FIG. 2 is a flow diagram of an implementation of a method of covering or camouflaging tissue according to the present invention;

FIG. 3 is a flow diagram of another implementation of a method of covering or camouflaging tissue according to the present invention;

FIG. 4A is a pictorial diagram of a portion of skin on a face of a patient, the skin including an unwanted visual characteristic; and

FIG. 4B is a pictorial diagram of a modified image of the portion of skin shown in FIG. 4A wherein the unwanted visual characteristic is removed.

### DETAILED DESCRIPTION OF THE INVENTION

Reference will now be made in detail to the presently preferred embodiments of the invention, examples of which to the extent present may be illustrated in accompanying drawings. Wherever possible, the same or similar reference numbers are used in drawings and the description to refer to the same or like parts. It should be noted that any drawings presented are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by any appended additional disclosure (e.g., in claims format). It is to be understood and appreciated that the process steps and structures described or incorporated by reference herein do not cover a complete process flow for the implementations described herein. The present invention may be practiced in conjunction with various methods and devices that are conventionally used in the art, and only so much of the commonly practiced method steps and structures are included herein as are necessary to provide an understanding of the present invention.

Any feature or combination of features described herein are included within the scope of the present invention provided that the features included in any such combination are not mutually inconsistent as will be apparent from the context, this specification, and the knowledge of one of ordinary skill in the art.

The present invention relates to the covering of tissue patches (e.g., areas of hard or soft tissue) with coatings, which coatings bear images of the underlying tissue patches. In accordance with the present invention, methods and apparatuses (e.g., tissue coatings, carrier sheets, and structures for making and applying same) for facilitating the generation and modification of tissue coverings are disclosed. The tissue coverings can bear images (e.g., customized tissue images) of underlying patches of tissue over which the tissue coverings are disposed. The images can be computer generated, and the tissue patch or tissue patches can comprise one or more unwanted features or blemishes. Furthermore, the images of the tissue patch may differ from the tissue patch being covered by at least one visual, topographical or compositional characteristic. In typical implementations, the tissue patch can comprise an unwanted feature or blemish that is attenuated, altered or removed on or from the image of the tissue.

The coatings, which bear images of the underlying tissue, can comprise photo-bearing tissue coatings. Such photo-bearing tissue (e.g., skin) coatings or other biocompatible photo-bearing layers, and methods of generating and applying same, are disclosed herein. The photo-bearing layers are suitable for application to an epidermis of a user, such as a blemish area of a user's face. Each photo-bearing layer may comprise coatings of color that are painted or otherwise transferred onto an epidermis of a user. One form of being otherwise transferred may comprise application by way of an adhesive structure such as, such as, for example, a BAND-AID® brand adhesive strip or other covering, temporary or permanent, single or multi-layered, as broadly defined in the art. Another form of being otherwise applied may comprise the transferring of one or more colors to the tissue (e.g., skin) in the form of a full (continuous) or partial coating (e.g., any type of color applied to the tissue such as skin or tattoo, temporary or permanent, single or multi-layered, as broadly defined in the art), wherein the photo-bearing layer is colored to mimic or blend-in with an image that has been previously captured of the user's tissue over which the photo-bearing layer is applied.

A photo-bearing tissue coating of the present invention can be based or derived, at least in part, from one or more images or likenesses of, or resembling, a patch of tissue (e.g., skin). The patch of tissue may belong to the user or ultimate recipient of the photo-bearing tissue (e.g., skin) coating, or may belong to another person, or may comprise a fictitious or simulated patch of tissue. Moreover, the patch of tissue may correspond to an anatomical location or area over which the photo-bearing tissue coating is to be placed. Such correspondence may comprise one or more of a texture or appearance thereof, topography, pattern, color variegation, or other visual characteristic or feature of the patch of tissue (e.g., skin).

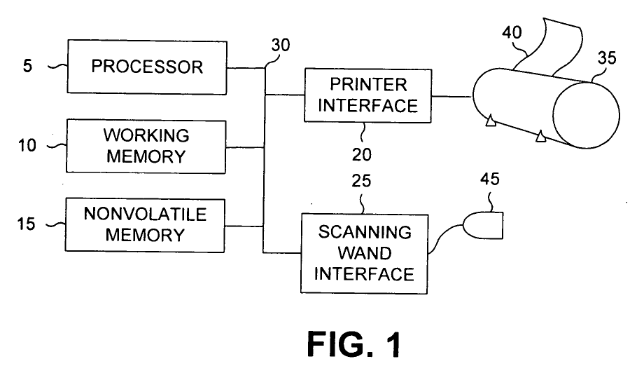

A block diagram of an embodiment of a device for treating a target area of tissue is illustrated in FIG. 1. The embodiment shown can comprise a computing device including, for example, a processor 5, a working memory 10 accessible by the processor 5, and a nonvolatile memory 15 which may, according to an exemplary embodiment, comprise instruction sequences capable of causing the processor 5 to perform functions according to the present invention. One or more of the mentioned elements may be implemented in a personal computer (PC) and/or may be implemented in a stand alone countertop system such as a wand-charging and/or sheet-printing box countertop device that is sold with or as part of the scanning wand 45.

The illustrated embodiment further can comprise an image-output interface such as a carrier printer interface 20, and an image acquisition device such as a scanning wand interface 25. The aforementioned elements can be interconnected by a system bus 30 and/or by one or more wireless protocols (e.g., BlueTooth® wireless technology) to facilitate intercommunication among the shown elements, and potentially among other elements such as, for example, in embodiments wherein image data for forwarding to the carrier printer interface is to be generated, edited or viewed by a user.

An image output device, such as a carrier printer 35 capable of printing images onto a carrier sheet 40, can be operatively connected to the carrier printer interface 20 so that the processor 5 is able to transfer an image to the carrier printer 35 for printing. An image acquisition device, such as a scanning wand 45, can be connected to the scanning wand interface 25 so that the scanning wand 45 can be controlled by the processor 5 in order to capture an image as described herein.

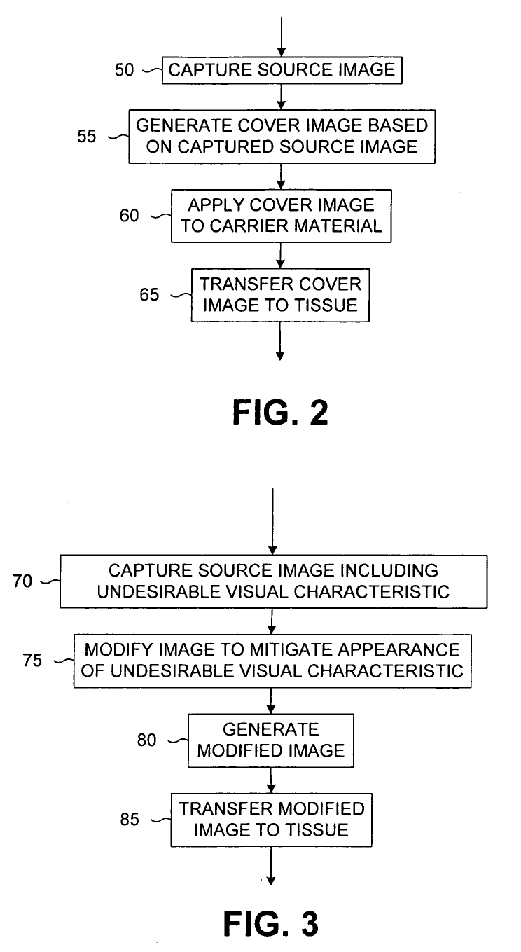

A flow diagram depicting an implementation of a method of covering or camouflaging tissue according to the present invention is shown in FIG. 2. According to the illustrated implementation, a source image is captured at step 50.

A. Source Image Capture (Step 50)

An exemplary implementation can comprise capturing a source image of tissue (e.g., skin). The source image can be taken from an area (e.g., patch) of soft tissue (e.g., skin or oral tissue comprising gingival tissue) or hard tissue (e.g., oral tissue comprising a tooth) of an individual being treated, such as, for example, a patient. In typical implementations of the present invention, the area can be located, for instance, on, in, or in a vicinity of any place of the body of the patient where wound treatments (e.g., bandages), other treatments or interventions (e.g., on the patient's skin such as varicose veins, bruises, birth marks, or in the patient's oral cavity such as tooth chips or discolorations, or periodontal undesired features or defects) have been applied, implemented, or observed.

The source image can be taken from an area of skin of another individual, or may generated based upon other source images or other sources or generated by other means, computer aided or otherwise. According to one implementation, a source image may be taken from an area of the body of a patient that is different from the area being treated. For example, in treating a blemish located, such as, for example, on the right side of the face of a patient, a source image of a corresponding portion of the (e.g., unblemished) left side of the patient may be captured. The capturing of the source image can comprise, for example, taking a relatively high-resolution photograph (as broadly used and understood in the art, such as greater than 1 megapixel, or even greater than 4 megapixels) of the user's skin. In other embodiments a relatively low-resolution (as broadly used and understand in the art, such as less than 0.5 or 1 megapixel) photograph is taken, and in still other embodiments other resolutions, defined for example, by pixel content, may be implemented. The photograph can comprise one or more color tones constituting a likeness of the user's skin. For example, the photograph may comprise two or more, such as, for example, 4 or 16, colors combined in such a way as to resemble a likeness of the user's skin in an area upon which the photo-bearing skin coating is to be applied. Of course, more typical embodiments can comprise more than two or four colors to generate more realistic appearing images of the user's skin. In further embodiments, many more than two colors, including even hundreds of colors, such as, for example, 256, 65,536 or 16,777,216, colors can be provided and used to generate the source image. Methods and apparatus for facilitating the acquisition and generation of digital images are well known in the art and incorporated herein by reference to any extent and in any combination and including any modifications to the extent not mutually exclusive. Such methods and apparatus can be applied, for example, to generate an image, preferably a digital image, that resembles the patch of skin of the user over which the photo-bearing skin coating is to be applied.

B. Image Generation to Treat Blemish (Step 55)

At step 55 of the implementation shown in FIG. 2, a modification of the source image captured at step 50 is generated to form a cover image. In a typical implementation of the present invention, a user may generate, modify, or approve a source image for application onto a patch of, for example, a patient's skin. Accordingly, an operator (e.g., the user) may load or execute a graphics software program onto or in connection with a personal home computer. The graphics software program can include a graphics library containing a predetermined number of pre-stored (e.g., pre-designed) graphic images and/or image editing or altering functions from which to choose. In addition, the graphics software program may include prompts as to the size of the photo-bearing skin coating sheet, the number and shape of the photo-bearing skin coatings on, for example, a carrier (e.g., carrier sheet), such as a bandage sheet, and may further include user-friendly prompts or guides to lead an operator through the modification (or direct transfer, without modification) and imprinting process. Modification of the source image may comprise any editing or altering of the source image, in any way known to those skilled in the art.

A typical modification may comprise the user editing a source image, which source image may contain a digital representation (e.g., digital high-resolution photograph) that resembles (e.g., that was acquired directly from, or that is a photograph or realistic reproduction of) a patch of skin of the user over which the photo-bearing skin coating is to be applied. Alternatively, relative to the example introduced above wherein an image of a portion of the (e.g., unblemished) left side of the face of a patient is captured, modification of the source image may comprise forming a mirror image of the captured image in order that the modified image may conform to a corresponding portion of the right side of the patient's face. Generally, a typical edit by the user may comprise the user modifying the source image, which is a digital photograph of the patch of skin of the user over which the photo-bearing skin coating will be applied, to remove a blemish. Blemishes may comprise any visual feature, such as a scar, birthmark, acme, or freckle, that the user wishes to visually modify or remove.

Methods and apparatus for facilitating the slight or substantial modification, in any way, such as, for example, the touching-up or resizing of a source digital image, are well known in the art and incorporated herein by reference to any extent and in any combination and including any modifications to the extent not mutually exclusive. The slight or substantial modifications to the source image may be accomplished utilizing a graphic workshop program embedded within the graphic software program. Moreover, modifications to the source image may be accomplished by the user on a home personal computer or may be accomplished at a point later in time at a remote location by, for example, other entities.

Following editing and/or designing of the source image, the resulting image (e.g., the unique or customized graphic image closely resembling the patch of skin of the user over which the photo-bearing skin coating will be applied to, for example, shield or conceal a blemish, located for example any place on the body (e.g., any place where bandages or other tissue treatments, such as wound treatments or oral hard or soft tissue treatments, have been applied) may be stored or forwarded directly to an output device for printing.

C. Application of Image to Carrier Material (Step 60)

The modified image may be applied to carrier material at step 60 of the implementation of the method of the present invention illustrated in FIG. 2. For example, an operator (e.g., the user) may load an appropriately sized carrier (e.g., bandage) sheet into a printer (e.g., a home printer), which is in data communication with the home personal computer, and print the modified image onto the sheet. The printing may be facilitated or implemented using any technique, structure and/or carrier material known in the art that can form, in whole or in part, the photo-bearing skin coating. A few examples of printing techniques, structures and materials, which are not intended to be limiting in any way in regards to options, can include printing with etched plates or cylinders such as intaglio printing (e.g., gravure), printing processes in which the image to be printed is rendered on a flat surface as on sheet zinc or aluminum and treated to retain ink while the nonimage areas are treated to repel ink (e.g., lithography), stencil methods of printmaking in which designs are imposed on a screen of silk or other fine mesh wherein blank areas are coated with an impermeable substance and ink is forced through the mesh onto the printing surface (e.g., screen), and printing on a rotary press employing water-based ink for printing images on as examples plastic, paper, or cardboard (e.g., flexography). Examples of applications of images to carrier materials are disclosed in U.S. Pat. Nos. 6,297,420, 6,455,752, and 6,512,160. In accordance with exemplary implementations, a user can take a digital photograph of a patch of tissue (e.g., soft tissue such as skin or hard tissue such as one or more teeth) on his or her face to thereby generate a source image, wherein the patch of tissue of the user corresponds to that over which a photo-bearing tissue (e.g., skin) coating will be applied. The source image is then imported into (e.g., manually or automatically, instantly or later in time) or simply held in (e.g., without importing in embodiments wherein the source image is initially generated within) a graphics software program. The graphics software program and/or an optional graphic workshop program may be utilized by the user to facilitate modification of the source image, wherein a typical edit may comprise the user at least partially attenuating, altering, deemphasizing, camouflaging, masking or removing a blemish (e.g., an unwanted or undesirable visual characteristic or feature such as a scar, wound, birthmark, acme, or freckle) within the source image. Methods of U.S. Pat. No. 6,512,160, for example, may then be implemented to transfer the source image (e.g., the modified source image) to the patch of tissue. Other examples of applications of images to carrier materials are disclosed, either expressly or inherently, for example, at http://www.bandaid.com/kids_prod.shtml. Regarding carrier materials, U.S. Pat. No. 5,586,971, which discloses a bandage with an irregular edge onto which a layer of makeup may be applied, may be implemented with or without the makeup or may be implemented with for example another material (e.g., part of the photo-bearing skin coating of a similar or different material or composition as more central parts of the photo-bearing skin coating encircled by the edge). Alternatively, the modified image may be printed directly onto, for example, the user's skin (infra) without any use or with attenuated use of a carrier material. Techniques, structures and/or carrier materials, in whole or in part, modified or not, of any of the above implementations can apply to any of the disclosure set forth herein, in any permutation or combination, to the extent compatible or not mutually exclusive or to the extent modifiable by one skilled in the art to be compatible or not mutually exclusive.

D. Transfer of Image to Skin (Step 65)

The image may be transferred to tissue at step 65 of the implementation of FIG. 2. According to an alternative implementation, step 60, as already described, is an optional step. In another alternative implementation, step 60 is performed as a part of step 65. That is, step 60 may a part of the same action as step 65 and/or may be performed before, after, or simultaneous in time with step 65. As mentioned, the carrier upon which a photo-bearing skin coating may be applied, can comprise, for example, a carrier sheet, such as a bandage sheet. A primary function of the carrier sheet may be to modify, conceal, camouflage, or otherwise alter a visual, compositional, or textual property of an area of hard or soft tissue, rather than to cover and protect a wound as may often be the traditional purpose of, for example, a bandage. Accordingly, to the extent a carrier sheet is implemented in the form of a bandage sheet, the bandage sheet may be embodied in a low-profile form, customized dimension (e.g., shape) and/or without an absorbent pad or any pad. In a typical implementation, a carrier sheet may take a form of a relatively small and circular (e.g., having a diameter of about 0.25 centimeters to about 1 centimeter) sheet having on one side a source image or modified image (e.g., photograph) of the user's skin at the application area (e.g., patch of tissue) of the sheet and having on the other (i.e., on an opposite) side an adhesive backing for being adhered to the area of application. Other exemplary constructions of carrier sheets may comprise, for example, any suitable shape, regular or irregular, and any size that may correspond to (e.g., be constructed to match) a shape or size of any area of the body where wound treatments (e.g., bandages), other treatments, or interventions (e.g., on the user's skin such as varicose veins, bruises, birth marks, or in the user's oral cavity such as tooth chips or discolorations, or periodontal undesired features or defects) have been applied, implemented, or observed.

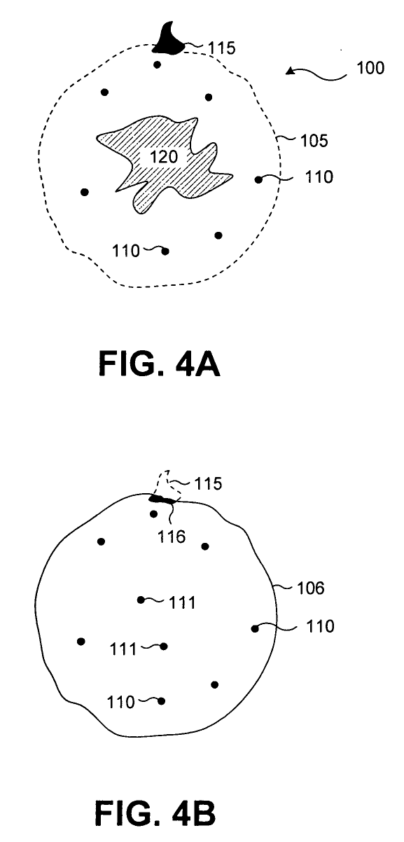

A flow sequence as presented in FIG. 3 elucidates another embodiment of the method of the present invention. According to this implementation, a source image of an application area (e.g., target area) of tissue is captured at step 70, wherein the target area may comprise an undesired visual characteristic. The scanning wand 45 (FIG. 1) may be employed to capture the source image. FIG. 4A illustrates an example of a portion 100 of skin on a face of a patient, the portion comprising, for example, a target area 105 shown in phantom in the figure. The illustrated portion 100 may comprise variations in color, such as small, natural, nominally circular freckles 110 as well as, for example, a relatively large normal freckle 115. The large normal freckle 115 is situated on a boundary of the target area 105 in the illustrated example. It may be desired to camouflage or mask an undesired freckle 120, which is shown near a center of the target area.

Returning to FIG. 3, the source image may be modified either automatically or manually as described herein to alter an appearance of (e.g., remove) the unwanted visual characteristic at step 75. With reference to FIG. 4A, the captured image may be edited either automatically or manually to remove the melonomic freckle 120 as illustrated in a modified version of the source image provided in FIG. 4B. It may be noted that the natural freckles 110 are retained in the modified image, according to the illustrated example, but need not be. Further, additional normal-appearing freckles 111 may be added to the modified image in order to enhance an effect of natural appearance. The modified image may also include a portion 116 of the relatively large normal freckle 115 (a portion of the large normal freckle 115 not within a border 106 of the modified image is shown in phantom in FIG. 4B) in order to facilitate matching skin features (e.g., coloration) at the border 106 of the modified image. A frontal image that maintains an appearance of the source image without the undesirable visual characteristic may be generated at step 80. In a modified embodiment, one or more additional undesirable characteristics may be altered or removed. According to an example of this embodiment, the additional characteristic may be portion 116 of the relatively large normal freckle 115.

According to the embodiment illustrated in FIG. 1, for example, the frontal image may be generated by causing the printer 35 (e.g., under control of the processor 5) to print the image on a carrier sheet 40 whence the image may be transferred to the target area at step 85 as has already been described. The carrier sheet may correspond, either partially or identically, to the size and/or shape of the captured image show in FIG. 4A, and further may correspond in coloration (and/or size, or shape) of the modified image shown in FIG. 4B.

Shapes or sizes of exemplary constructions of carrier sheets may be chosen or altered, using any technique or apparatus known to those skilled in the art, in order to, for example, modify, conceal, camouflage, or otherwise alter a visual, compositional, or textual property of an underlying area of hard or soft tissue. Accordingly, dimensions and/or surface properties of carrier sheets of the present invention will often correspond (e.g., match) dimensions and/or surface properties of the underlying hard or soft tissue areas being covered. The source image or modified image will typically correspond (e.g., match) one or more or all of the characteristics of the underlying patch of hard or soft tissue. In other implementations, a boundary (e.g., perimeter of) the source image or modified image will typically correspond (e.g., blend-in with or match) one or more or all of the characteristics of a boundary (e.g., perimeter of) the underlying patch of hard or soft tissue.

While the description above refers to particular embodiments of the present invention, it will be understood that many modifications may be made without departing from the spirit thereof. Aspects of the invention may have combinations of the above-described embodiments although these combinations may not be explicitly described. Any accompanying additional disclosure in claims format is intended to cover such embodiments as would fall within the true scope and spirit of the present invention.

1. A device for treating a target area of tissue, comprising:
a carrier constructed to be applied in proximity to and to at least partially cover the target area of tissue; and
a surface coupled to the carrier and constructed with an image of the target area of tissue.

2. The device as set forth in claim 1, wherein the device is adapted to treat oral tissue.

3. The device as set forth in claim 2, wherein the device is adapted to treat a tooth.

4. The device as set forth in claim 2, wherein the device is adapted to treat oral tissue comprising gingival tissue.

5. The device as set forth in claim 1, wherein the device is adapted to treat soft tissue comprising skin.

6. The device as set forth in claim 1, wherein the image of the target area of tissue comprises a modified image of the target area of tissue.

7. The device as set forth in claim 6, wherein the image of the target area of tissue comprises a modified digital photograph of the target area of tissue.

8. The device as set forth in claim 6, wherein the image of the target area of tissue comprises an image of the target area of tissue from which at least one unwanted feature has been removed.

9. The device as set forth in claim 1, wherein the carrier comprises an adhesive structure.

10. A device for covering an unwanted feature in an application area of tissue of a patient, the device comprising:
a first side having disposed thereon a modified image of the application area; and
a second side having disposed thereon an adhesive backing adapted to being adhered to a vicinity of the application area.

11. The device as set forth in claim 10, wherein the modified image comprises a digital photograph of the application area in which the unwanted feature has been attenuated, altered, and removed.

12. The device as set forth in claim 10, wherein the device comprises an adhesive strip.

13. The device as set forth in claim 10, wherein the carrier sheet comprises a circular shape having a diameter ranging from about 0.25 centimeter to about 1 centimeter.

14. The device as set forth in claim 10, wherein the carrier sheet comprises an irregular shape which corresponds to a shape of the unwanted feature.

15. A method of covering a target area of tissue, comprising:
capturing a source image;
generating a modification of the source image to create a frontal image which resembles the source image in appearance; and
transferring the frontal image to the target area of tissue.

16. The method as set forth in claim 15, wherein the transferring comprises:
transferring the frontal image to carrier material; and
applying the carrier material to the target area of tissue.

17. The method as set forth in claim 16, wherein the capturing comprises forming a digital photograph.

18. The method as set forth in claim 17, wherein the capturing further comprises forming a digital photograph of the target area of tissue.

19. The method as set forth in claim 18, wherein:
the digital photograph comprises at least one blemish present in the target area of tissue; and
the generating comprises modifying the source image to accomplish at least one of attenuating, altering, and removing of the at least one blemish.

20. The method as set forth in claim 16, wherein the transferring of the frontal image to carrier material comprises printing the frontal image on the carrier material.

21. The method as set forth in claim 16, wherein the carrier material comprises an adhesive structure.

22. The method as set forth in claim 15, wherein:
the source image has at least one undesirable visual characteristic;
the generating comprises modifying a part of the source image that corresponds to the undesirable visual characteristic to at least partially deemphasize, camouflage, mask or remove the undesirable visual characteristic, while maintaining a general appearance of the application area surrounding the undesirable visual characteristic.

23. The method as set forth in claim 22, wherein the generating further comprises using the source image and the modified part of the source image to create the frontal image.
